(19) United States
(12) Patent Application Publication
(10) Pub. No.: US 2004/0208916 A1
(43) Pub. Date: Oct. 21, 2004

(54) Patch for the transdermal administration of beneficial substances

(76) Inventors: Nick Abbott, Sheffield (GB); Jonathan Bowtell, Sheffield (GB)

Correspondence Address: Jonathan P. Osha, ROSENTHAL & OSHA L.L.P., Suite 2800, 1221 McKinney Street, Houston, TX, 77010, US

(21) Appl. No.: 10/414,416

(22) Filed: Apr. 15, 2003

(51) Int. Cl. A61K 9/70; A61K 35/20

(52) U.S. Cl. 424/449; 424/535

(57) ABSTRACT

A patch for the delivery of substances to the skin of a user, said patch comprising an impermeable substrate comprising an upper and a lower surface, the lower surface comprising an adhesive configured to substantially adhere the patch to the skin of a user; and the lower surface further comprising a matrix containing such substances, the patch being characterised in that it comprises colostrum and/or a colostrum derivative and/or a multi-vitamin and mineral solution.

### FIELD OF THE INVENTION

The present invention relates to a device and method for the transdermal administration of colostrum and/or a derivative of colostrum and/or a multi-vitamin and mineral solution to a user.

### BACKGROUND TO THE INVENTION

It is known that colostrum is the mammary secretion that a mammal produces for its newborn infant in the first 24 to 48 hours following birth. It contains numerous immune system and growth factors as well as essential nutrients. It is estimated that colostrum triggers at least 50 important processes in the newborn. Clinical studies have shown that bovine colostrum is biologically transferable to all mammals, including man and is much higher in immune factors than human colostrum. Studies indicate that all species, including man, benefit from the immunogenic properties of bovine colostrum with no reports of adverse reactions to date.

Historically, Ayurvedic physicians have used bovine colostrum therapeutically in India for thousands of years. In the US and throughout the world, conventional doctors used it for antibiotic purposes prior to the introduction of modern sulfur drugs and penicillin. In the early 1950's, colostrum was prescribed extensively for the treatment of rheumatoid arthritis. In 1950, Doctor Albert Sabin, the developer of the vaccine for polio, discovered that colostrum contained antibodies against polio and recommended it's use for children susceptible to catching the disease.

Existing methodologies for the administration of colostrum involve taking 1000 to 2000 milligrams of dried colostrum powder twice daily best taken on an empty stomach with a glass of water. Through hundreds of years of use and over 1000 clinical studies, colostrum has been demonstrated to be completely safe without any known drug interactions or side effects at any nominal level of ingestion.

In addition to oral administration of colostrum, previous workers in this field have demonstrated the usefulness of colostrum in the form of an eye drop, lotion cream or ointment for the treatment of dermatological or opthalmological conditions, for instance UK Patent application number 2052979. The following clinical conditions have been shown to respond favourably to colostrum administration: viral illnesses, allergies and auto-immune diseases, heart disease, cancer, diabetes, clinical obesity, athletic stress, leaky gut syndrome and wound healing.

The best forms of bovine colostrum come from cows which are raised organically in an environment free of pesticides, herbicides and fed upon feed stuffs lacking anabolic steriods, artificial growth factors, antibiotics and other chemical supplements.

The most important components of colostrum can basically be broken down into two major categories: immune system factors and growth factors. Drug manufacturers have tried to copy (genetically engineer) and market several of the individual components of colostrum, most notably inteferon, gamma globulin, growth hormone, IGF-1 and protease inhibitors.

The known components of colostrum will now be listed together with some discussion of their effects:

Immunogoblins, also called antibodies, neutralise pathogens in the lymphatic and circulatory systems. Many people take colostrum for its immunogoblins in an effort to receive passive immunity for a wider range of pathogens.

Lactoferrin is an iron binding protein that aids the body in utilising iron. The ability of lactoferrin to bind iron is important in keeping invading bacteria in check as they require iron to multiply. Many other functions have been attributed to lactoferrin, including more general antimicrobial and antiviral activities, immune regulation and cell growth regulation. Lactoferrin also enhances phagocytosis (the engulfing of harmful organisms by white blood cells), and has a powerful role in reducing the inflammation that accompanies many health problems.

Transferin is another mineral binding carrier protein that attaches to available iron and can act independently or in concert with lactoferrin to impede the growth of certain aerobic bacteria, most particularly in the gut.

Lysozyme is an enzyme that can breakdown the outer cell wall of certain bacteria, thus inhibiting their reproduction and making them vulnerable to attack by other immune factors. Found most predominantly in saliva, tears and in other muscosal membranes. Lysozyme plays a key role as first line of defence against bacteria and is so important that it has recently been added to several baby formulas in an effort to boost infant immunity.

Peroxidase is an enzyme that generates the release of hydrogen peroxide, this reactive chemical causing great damage to surrounding bacteria and other invading organisms.

Proline rich polypeptide (PRP) is a hormone that helps regulate the immune system, maintaining homeostasis in this important biological system. This regulation is extremely important for people with autoimmune diseases and it has been shown that proline rich polypeptide has a balancing effect on the immune response and can suppress an overactive immune system in cases where the immune system has begun to attack the healthy tissues of the body. It is also a powerful anti-inflammatory agent which has in some cases been shown to relieve pain.

Cytokines are messenger proteins that mediate many immune functions. They are involved in the production of t cells, lympathic activity and in regulating the severity and duration of the bodies immune response. One cytokine, known as interleukin 10, is highly anti-inflammatory and is a novel form of relieving discomfort among people with arthritis related pain.

Lymphokines are hormone like peptides. They are released by activated white blood cells and help to regulate the immune response.

Oligo saccharides and glycoconjugates bind to the surface of the intestines and other surfaces preventing the attachment of microorganisms. Evidence is also available that these components act as growth promoters for beneficial flora in the gastrointestinal tract and other areas of the body which have an innate bacterial flora.

Thymosin is a hormone composed of two protein based chains known as the alpha and beta chain which are separately present in colostrum. The chains act upon the thymus glad independently or together to stimulate activation, development and maintenance of the immune system.

Transfer factors are small proteins produced in response to the bodies exposure to types of microorganisms, particularly those that reside in deep tissues for a long period of time, like the bacteria which causes tuberculosis. They are specific for a particular microorganism and are carried inside certain types of specialised white blood cells. Transfer factors work together with various white blood cells and other factors in an attempt to keep pathegenic microorganisms under control.

Nucleotides and nucleosides are important for many metabolic functions, they enhance antibody response and contribute to absorption during digestion.

Xanthine oxidase is an enzyme that can attach to the cell walls of certain bacteria and interfere with the ability of the bacteria to replicate itself.

Growth hormone is the single most abundant hormone produced by the mammalian body and effects almost every cell. Growth hormone levels are highest during teenage years and fall rapidly thereafter. Growth hormone increases metabolism, reduces the laying down of fat and increases muscle mass. It is also believed to be involved in the regeneration of heart, lung and liver tissue as well as many other organs and tissues throughout the body. Growth hormone stimulates protein synthesis which is critical for the renewal of skin and bones. It is also considered to be an immune stimulant because it helps the body to produce antibodies, t cells and other types of white blood cells. Growth hormone has even been shown to effect the activity of neurotransmitters in the brain improving moods and overall activity.

Insuline like growth factors (IGF) I and II belong to a whole family of hormones contained in colostrum, called the IGF super family. IGF I is considered to be the most important of these, triggering the events that activate cell growth and reproduction, protein synthesis and the release of energy (glucose metabolism). It is also involved in many other major bodily functions and IGF I is found in association with almost all the cells of the body.

Epithelial growth factor stimulates normal skin growth.

Transforming growth factors A and B are helpful in healing wounds and in the synthesis and repair of RNA and DNA.

Fibroblast growth factor stimulates the growth of new blood vessels and contributes to tissue development and wound healing.

Platelet-derived growth factor is involved in the healing of vascular wounds. It is released in conjunction with blood clotting during the healing process.

Trypsin inhibitors and other protein inhibitors help the digrestion and uptake of immune and growth factors and also help prevent bacteria such as H. phylon from attaching to the walls of the stomach.

Leptin is a small hormone like protein that can suppress appetite and lead to body weight reduction. Mature fat cells (adipocytes) release leptin in the presence of insulin which is also found in collostrum. Insulin producing pancreatic beta cells have characterised binding sites for leptin and it is believed that the size of fat cells may be a major factor in determining the amount of leptin released. A deficiency of leptin may therefore be associated with obesity, particularly in diabetic individuals.

Insulin is a hormone required for the effective utilisation of glucose in the body. Insulin binds to specific sites on cells, facilitating their interaction with IGF I and thus initiating the conversion of glucose to glycogen, a high energy but stable carbohydrate.

Vitamin binding proteins act as stores and carriers to deliver vitamin B complexes throughout the body as well as acting as the carriers of other vitamins.

Mineral binding proteins especially lactoferrin and tranferrin not only interfere with the replication of certain pathogenic microorganisms; they also serve to capture iron and present it a form that can be readily absorbed and used by the body. Lactoferrin may also bind copper and delivery it in a form for absorption and use by the body. In addition two carrier proteins are also present in colostrum, these assist in calcium absorption and are called casein and alpha-lactalbumin.

Cyclic adenosine monophosphate (CAMP) is a phosphoralated nucleotide in a very specialised form that transfers and stores the chemical energy necessary to drive metabolic reactions to form new proteins, carbohydrates and fat molecules in the body.

Enzyme inhibitors, also known as permeability factors, are actually small proteins that slow down or inhibit the enzymatic breakdown of proteins increasing the biological half life of such proteins.

In addition to the benefits of taking colostrum, the benefits of maintaining the recommended daily intake of the major vitamin and minerals required by the body to function are well known and the use of vitamin and mineral supplements is also known and an accepted way of maintaining the levels of these important substances. Such multi-vitamin and mineral supplements are generally taken orally once or twice a day.

The inventors recognising the beneficial effects of administration of colostrum, a colostrum derivative or a multi-vitamin and mineral solution decided to attempt to improve and make easier the administration of these substances by incorporating them into a transdermal patch. The skin provides an ideal system for the delivery of agents systemically. Although the skin provides the body with a protective covering, a number of agents are absorbable through the skin and since the skin is highly vascularised, such agents are absorbed directly by the circulating blood supply and are therefore distributed immediately throughout the body, ultimately pervading the whole body.

It is therefore an object of the present invention to provide a transdermal patch delivery system for the administration of colostrum, one of colostrums derivatives or a multi-vitamin and mineral solution.

### SUMMARY OF THE INVENTION

According to a first aspect of the present invention there is provided a patch for the delivery of substances to the skin of a user, said patch comprising:

an impermeable substrate comprising an upper and a lower surface;

said lower surface comprising an adhesive configured to substantially adhere said patch to the skin of a user; and

said lower surface further comprising a matrix containing said substances;

said patch being characterised in that said substances comprise colostrum.

According to a second aspect of the present invention there is provided a patch for the delivery of substances to the skin of a user, said patch comprising:

an impermeable substrate comprising an upper and a lower surface;

said lower surface comprising an adhesive configured to substantially adhere said patch to the skin of a user; and

said lower surface further comprising a matrix containing said substances; said patch being characterised in that said substances comprise a derivative of colostrum selected from the group comprising:

immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

According to a third aspect of the present invention there is provided a patch for the delivery of substances to the skin of a user, said patch comprising:

an impermeable substrate comprising an upper and a lower surface;

said lower surface comprising an adhesive configured to substantially adhere said patch to the skin of a user; and

said lower surface further comprising a matrix containing said substances;

said patch being characterised in that said substances comprise a multi-vitamin and mineral solution.

According to a fourth aspect of the present invention there is provided a method for manufacturing the patch for the transdermal administration of colostrum comprising the steps:

extruding a sheet of an impermeable substrate;

applying a layer of adhesive to one surface of said sheet;

forming a matrix configured to store colostrum upon said surface;

impregnating said matrix with colostrum;

covering said surface with a removable film.

According to a fifth aspect of the present invention there is provided a method for manufacturing the patch for the transdermal administration of a derivative of colostrum comprising the steps:

extruding a sheet of an impermeable substrate;

applying a layer of adhesive to one surface of said sheet; forming a matrix configured to store a derivative of colostrum upon said surface;

impregnating said matrix with a derivative of colostrum; covering said surface with a removable film.

According to a sixth aspect of the present invention there is provided a method for manufacturing the patch for the transdermal administration of a multivitamin and mineral solution comprising the steps:

extruding a sheet of an impermeable substrate;

applying a layer of adhesive to one surface of said sheet;

forming a matrix configured to store a multivitamin and mineral solution upon said surface;

impregnating said matrix with a multivitamin and mineral solution;

covering said surface with a removable film.

### BRIEF DESCRIPTION OF THE DRAWINGS

For a better understanding of the invention and to show how the same may be carried into effect, there will now be described by way of example only, specific embodiments, methods and processes according to the present invention with reference to the accompanying drawings in which:

FIG. 1 shows a schematic and cut through representation of a transdermal patch according to a first specific embodiment of the present invention.

FIG. 2 shows a schematic and cut through representation of a transdermal patch according to a second specific embodiment of the present invention.

### DETAILED DESCRIPTION OF A SPECIFIC MODE FOR CARRYING OUT THE INVENTION

There will now be described by way of example a specific mode contemplated by the inventors. In the following description numerous specific details are set forth in order to provide a thorough understanding. It will be apparent however, to one skilled in the art, that the present invention may be practiced without limitation to these specific details. In other instances, well known methods and structures have not been described in detail so as not to unnecessarily obscure the description.

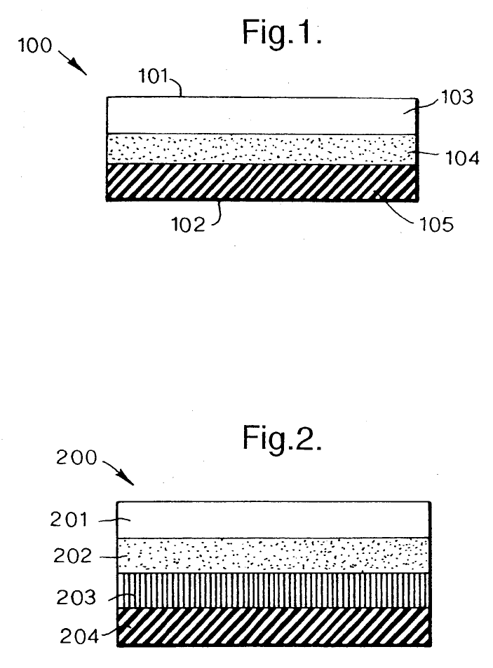

According to a first specific embodiment of the present invention and referring to FIG. 1 there is provided a patch for the transdermal administration of colostrum 100. This patch 100 comprises an upper surface 101 and a lower surface 102. The patch further comprises an impermeable substrate 103 forming the upper surface 101, onto a lower surface of this impermeable substrate 103 is applied a layer of adhesive 104 and onto this layer of adhesive is applied a removable covering material 105 which forms the lower surface 102 of the patch 100. In this specific embodiment of the present invention the impermeable substrate comprises PVC but any suitable impermeable material may be used. The adhesive 104 comprises an acrylic ester copolymer impregnated with colostrum. In alternative embodiments of the present invention the adhesive 104 may comprise any adhesive selected from the group: acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

Colostrum may be applied to the adhesive 104 either during the preparation of the adhesive prior to its application onto the impermeable substrate 103 or alternatively colostrum in the form of a liquid spray or powder may be applied to the adhesive layer 104 following its application onto the impermeable substrate 103, before the removable material 105 is applied.

Alternatively, in place of colostrum a known derivative of colostrum selected from the group comprising: immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

Or alternatively a multi-vitamin and mineral solution may instead be incorporated into the adhesive 104, giving rise to a colostrum derivative patch or a multi-vitamin and mineral patch configured to transdermally administer these beneficial substances.

In operation the patch 100 may be used as follows:

The patch 100 would be stored in an airtight container in order to maintain the active ingredients in the patch 100, in as optimal a condition as possible for the greatest time. When such a patch was to be used, this airtight packaging would be removed and the removable covering material 105 would be peeled back from the underside of the patch 100. The user of the patch may then place the patch, adhesive 104 side down, onto their skin in a position where the patch will not be disturbed and leave the patch in this position until the patch is depleted of compound substances to be administered transdermally. At this time the patch may simply be removed from the skin of the user and replaced with a fresh patch much as previously described.

A second preferred embodiment of the present invention is shown herein in FIG. 2. There is provided a patch 200 for the delivery of substances to the skin of a user which comprises an impermeable substrate 201, this impermeable substrate 201 comprises an upper and a lower surface and upon the lower surface of this impermeable substrate there is a layer of adhesive 202 configured to substantially adhere the patch 200 to the skin of a user. The patch 200 further comprises a piece of gauze material 203 adhered to the layer of adhesive 202 but of smaller dimension that the impermeable substrate 201, such that areas of the impermeable substrate 201 covered with adhesive 202 at the periphery of the gauze material 203 continue to be exposed to the environment. Finally, a layer of a removable covering material 204 is adhered to the exposed areas of adhesive such that the gauze material 203 is enclosed within an airtight cavity. In this specific embodiment the impermeable substrate comprises polypropylene, although any suitable impermeable polymer such as PVC may also be used. In the specific embodiment of the present invention the adhesive 202 comprises a silicone adhesive suitable for use in contact with human skin. The gauze material 203 comprises a natural cotton fibre with suitable water absorption qualities. Alternatively, any other form of natural or man-made fibre may be used. In this specific embodiment of the present invention this cotton gauze 203 is impregnated with a solution comprising colostrum. Colostrum may be applied to this gauze material in the form of the solution either prior to the gauzes incorporation into the patch 200 or a suitable colostrum solution or colostrum in a powder form may be directly applied to the gauze material 203 following the incorporation of the gauze material 203 into the patch 200.

Alternatively, the substance to be administered may be incorporated into a matrix comprising a thickening agent. This thickening agent comprising a natural or synthetic gel-forming polymer selected from the group consisting of gum karaya, gum acacia, locust bean gum, guar gum, modified guar gum, maltodextrin, carboxymethyl cellulose, carboxypropyl cellulose, poly-acrylamide, poly acrylic acid, a natural or synthetic rubber, a petroleum derivative, and a resin. Such a formulated gel being used in place of the gauze material in this second embodiment.

Alternatively, in place of colostrum, a known derivative of colostrum or a multi-vitamin and mineral solution may instead be incorporated into the gauze material 203 much as previously described for the first specific embodiment of the present invention.

This second specific embodiment of the present invention may be used in a similar manner to that previously described in the first specific embodiment of the present invention.

Details relating to the collection, purification and formulation of solutions and powders comprising colostrum or a derivative of colostrum are standard throughout the field. Details relating to the formulation and production of multi-vitamin and mineral solutions are standard throughout the field and do not affect the character or implementation of the present invention.

1. A patch for the delivery of substances to the skin of a user, said patch comprising:
an impermeable substrate comprising an upper and a lower surface;
said lower surface comprising an adhesive configured to substantially adhere said patch to the skin of a user; and
said lower surface further comprising a matrix containing said substances;
said patch being characterised in that said substances comprise colostrum.

2. A patch as claimed in claim 1, wherein said impermeable substrate comprises a polymer.

3. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

4. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

5. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a multi-vitamin and mineral solution.

6. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises said adhesive and said substances.

7. A patch is claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises a gauze and said substances.

8. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises said adhesive and said substances.

9. A patch as claimed in claim 1, wherein said impermeable substrate comprises a polymer, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises a gauze and said substances.

10. A patch as claimed in claim 1, wherein said impermeable substrate comprises PVC.

12. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

13. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

14. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution.

15. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises said adhesive and said substances.

16. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises a gauze and said substances.

17. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises said adhesive and said substances.

18. A patch as claimed in claim 1, wherein said impermeable substrate comprises PVC, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises a gauze and said substances.

19. A patch as claimed in claim 1, wherein said impermeable substrate comprises polypropylene.

20. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

21. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

22. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution.

23. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises said adhesive and said substances.

24. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors;
wherein said matrix comprises a gauze and said substances.

25. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises said adhesive and said substances.

26. A patch as claimed in claim 1, wherein said impermeable substrate comprises polypropylene, and wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and
wherein said substance further comprises a multi-vitamin and mineral solution;
wherein said matrix comprises a gauze and said substances.

27. A patch as claimed in claim 1, wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin; and

28. A patch as claimed in claim 1, wherein said substances further comprise a derivative of colostrum selected from the group comprising:
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

29. A patch as claimed in claim 1, wherein said substances further comprise a multi-vitamin and mineral solution.

30. A patch as claimed in claim 1, wherein said matrix comprises said adhesive and said substances.

31. A patch as claimed in claim 1, wherein said matrix comprises a gauze and said substances.

32. A patch for the delivery of substances to the skin of a user, said patch comprising:
an impermeable substrate comprising an upper and a lower surface;
said lower surface comprising an adhesive configured to substantially adhere said patch to the skin of a user; and
said lower surface further comprising a matrix containing said substances;
said patch being characterised in that said substances comprise a derivative of colostrum selected from the group comprising
immunoglobulins, lactoferin, transferin, lysozyme, peroxidase, proline rich polypeptides, cytokines, lymphokines, oligo saccharides and glycoconjugates, thymosin, transfer factors, nucleotides and nucleosides, xanthine oxidase, growth hormone, insulin-like growth factors, epithelial growth factors, transforming growth factors A and B, fibroblast growth factor, platelet derived growth factors, trypsin inhibitors, protease inhibitors, leptin, insulin, vitamin—binding proteins, mineral binding proteins, cyclic adenosine monophosphate, enzyme inhibitors.

33. A patch as claimed in claim 32, wherein said impermeable substrate comprises a polymer.

34. A patch as claimed in claim 32, wherein said impermeable substrate comprises PVC.

35. A patch as claimed in claim 32, wherein said impermeable substrate comprises polypropylene.

36. A patch as claimed in claim 32, wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

37. A patch as claimed in claim 32, wherein said matrix comprises said adhesive and said substances.

38. A patch as claimed in claim 32, wherein said matrix comprises said adhesive and said substances.

39. A patch as claimed in claim 32, wherein said matrix comprises gauze and said substances.

40. A patch for the delivery of substances to the skin of a user, said patch comprising:
an impermeable substrate comprising an upper and a lower surface;
said lower surface comprising an adhesive configured to substantially adhere said patch to the skin of a user; and
said lower surface further comprising a matrix containing said substances;
said patch being characterised in that said substances comprise a multi-vitamin and mineral solution.

41. A patch as claimed in claim 40, wherein said impermeable substrate comprises a polymer.

42. A patch as claimed in claim 40, wherein said impermeable substrate comprises PVC.

43. A patch as claimed in claim 40, wherein said impermeable substrate comprises polypropylene.

44. A method for manufacturing the patch for the transdermal administration of colostrum comprising the steps:
extruding a sheet of an impermeable substrate;
applying a layer of adhesive to one surface of said sheet;
forming a matrix configured to store colostrum upon said surface;
impregnating said matrix with colostrum;
covering said surface with a removable film.

45. A method as claimed in claim 44 for manufacturing a patch for the transdermal administration of colostrum wherein said impermeable substrate is a polymer.

46. A method as claimed in claim 44 for manufacturing a patch for the transdermal administration of colostrum wherein said impermeable substrate is PVC.

47. A method as claimed in claim 44 for manufacturing a patch for the transdermal administration of colostrum wherein said impermeable substrate is polypropylene.

48. A method a claimed in claim 44 for the manufacturing of a patch for the transdermal administration of colostrum wherein said adhesive is selected from the group comprising:
acrylate emulsion adhesive, an acrylic ester copolymer, a vinyl acetate resin, a copolymer of vinyl acetate and dioctyl maleate, silicone adhesive, natural or synthetic rubber, a petroleum derivative, a resin.

49. A method as claimed in claim 44 for the manufacturing of a patch for the transdermal administration of colostrum wherein said matrix comprises said adhesives.

50. A method as claimed in claim 44 for the manufacturing of a patch for the transdermal administration of colostrum wherein said matrix comprises a gauze material.

51. A method as claimed in claim 44 for the manufacturing of a patch for the transdermal administration of a colostrum derivative wherein said matrix comprises said adhesive.

52. A method as claimed in claim 44 for the manufacturing of a patch for the transdermal administration of a colostrum derivative wherein said matrix comprises a gauze material.

53. A method as claimed in claim 44 for the manufacturing of a patch for the transdermal administration of a multi-vitamin and mineral solution wherein said matrix comprises said adhesive.

54. A method as claimed in claim 44 for the manufacturing of a patch for the transdermal administration of a multi-vitamin and mineral solution wherein said matrix comprises a gauze material.

55. A method for manufacturing the patch for the transdermal administration of a derivative of colostrum comprising the steps:
extruding a sheet of an impermeable substrate;
applying a layer of adhesive to one surface of said sheet;
forming a matrix configured to store a derivative of colostrum upon said surface;
impregnating said matrix with a derivative of colostrum;
covering said surface with a removable film.

56. A Method for manufacturing the patch for the transdermal administration of a multivitamin and mineral solution comprising the steps:
extruding a sheet of an impermeable substrate;
applying a layer of adhesive to one surface of said sheet;
forming a matrix configured to store a multivitamin and mineral solution upon said surface;
impregnating said matrix with a multivitamin and mineral solution;
covering said surface with a removable film.
